(19) United States
(12) Patent Application Publication
(10) Pub. No.: US 2008/0014177 A1
(43) Pub. Date: Jan. 17, 2008

(54) Devices, methods, and systems for accessing native neurons through artificial neural mediators (ANMs)

(75) Inventors: James R. Kozloski, New Fairfield, CT (US); Stanislav Polonsky, Putnam Valley, NY (US)

Correspondence Address: SCULLY SCOTT MURPHY & PRESSER, PC, 400 GARDEN CITY PLAZA, SUITE 300, GARDEN CITY, NY, 11530, US

(73) Assignee: International Business Machines Corporation, Armonk, NY (US)

(21) Appl. No.: 11/487,810

(22) Filed: Jul. 17, 2006

(51) Int. Cl. A61K 35/30; C12N 5/08

(52) U.S. Cl. 424/93.7; 435/368

(57) ABSTRACT

The present invention relates to devices, methods, and systems for accessing native neurons in the nervous system of an animal. Specifically, one or more artificial neural mediators (ANMs) each comprising a neural cell are first formed, and neural connection is then established between the ANMs and one or more native neurons or collections of native neurons located in the nervous system. In this manner, the native neurons or collections of native neurons can be assessed through the ANMs. The neural connection between the ANMs and the native neurons is preferably established by guided axonal growth in the present invention, i.e., either an axon from one of the ANMs is grown into contact with one of the native neurons or collections of native neurons, or an axon from one of the native neurons or collections of native neurons is grown into contact with one of the ANMs.

### FIELD OF THE INVENTION

The present invention generally relates to devices, methods, and systems for accessing native neurons in the nervous system of an animal for the purposes of recording from the native neurons, applying artificial stimuli to the native neurons, or delivering chemical or biological materials to the native neurons. More specifically, the present invention relates to devices, methods, and systems that employ artificial neural mediators for accessing the native neurons in the nervous system in a neuron-specific and non-invasive manner.

### BACKGROUND OF THE INVENTION

The nervous system of an animal (including humans) is composed of billions of neurons that communicate with each other via patterns of action potentials relayed across a biological neural network, which comprises anatomically specified connections formed between such neurons.

Many highly specialized types of neurons exist, which may differ widely in appearance. Regardless of its cell type, each neuron comprises the following components:
(1) a cell body, which is the central part of the neuron where the nucleus is located and where most protein synthesis occurs;
(2) multiple dendrites, which are branches or cell extensions formed around the cell body and which function as the main information-receiving components of the neuron;
(3) a single axon, which is a long, cable-like projection that may extend tens of thousands of times the diameter of the cell body in length and which functions as the main information-outputting component of the neuron; and
(4) an axon terminal, which is located at the end of the axon away from the cell body. The axon may undergo extensive branching (i.e., form axon collaterals) to thereby enable the single axon to connect to multiple axon terminals and multiple other neurons.

The interface through which a specific neuron interacts with surrounding neurons in a biological neural network consists of: (1) the multiple dendrites of the specific neuron, which are connected to multiple up-stream neurons and function as input connections from the upstream neurons to the specific neuron, and (2) the single axon of the specific neuron, which is connected to one or more down-stream neurons and functions as an output connection from the specific neuron to the one or more down-stream neurons. Input signals from the multiple up-stream neurons will summate in the cell body of the specific neuron (which is commonly referred to, as neural summation), and when the sum of such input signals surpasses a certain threshold, an action potential will be generated by the specific neuron and sent through the axon of the specific neuron to the one or more down-stream neurons.

Neurons communicate with one another through synapses, which are junctions across which nerve impulses pass from an axon terminal of a pre-synaptic neuron to a post-synaptic neuron. The nerve impulses are typically passed between neurons by chemicals such as neurotransmitters. The detailed steps of a chemical-based synapse include: when an action potential reaches the axon terminal of the pre-synaptic neuron, the wave of changing charges opens voltage-gated calcium channels at the axon terminal, thus allowing calcium ions to enter the pre-synaptic neuron at the axon terminal. Calcium causes synaptic vesicles that are located inside the pre-synaptic neuron and contain neurotransmitter molecules to fuse with the membrane of the pre-synaptic neuron at the axon terminal, thereby causing release of the neurotransmitters into the synaptic cleft located between the pre-synaptic neuron and the post-synaptic neuron. The released neurotransmitters diffuse across the synaptic cleft and activate receptors on the post-synaptic neuron. Nerve impulses can also be passed between neurons in the form of electrical pulses through direct, electrically conductive junctions.

Easy access to native neurons in the nervous systems is crucial for neurological studies and treatments. On one hand, in order to fully understand how action signals are generated and transmitted in the nervous systems, one has to be able to monitor native neurons and record neural activities of such native neurons on a temporal basis. The recordings from the native neurons can then be analyzed to determine correlations, interactions, communications and information processing mechanisms implemented by the nervous systems. On the other hand, artificial stimulations of native neurons at specific regions of the nervous system have played an important role in neurological rehabilitation. Artificial stimulations, when carefully applied, can be effective in restoring certain lost or impaired neurological functions that are controlled by the specific regions of the nervous system.

Microelectrodes have been used conventionally for accessing native neurons in the nervous system. Such microelectrodes are inserted into a specific region in the nervous system to contact the native neurons located at the specific region, and electrical input or output signals can then be delivered to, or received from, the native neurons through such microelectrodes.

Ideally, a specific one-to-one contact between a microelectrode and a native neuron or native neuron type is needed to achieve neuron-specific recording and stimulation. However, due to the fact that the currently available microelectrodes are directed only to specific brain regions, and cannot be directed to specific neurons or neuron types within said regions, an inserted or implanted microelectrode inevitably stimulates at random one or many native neurons within the targeted region. Therefore, a specific one-to-one contact between a microelectrode and a native neuron or native neuron type is currently impossible.

Insertion of the microelectrodes into the nervous system is invasive, especially when the insertion site is located deep inside the nervous system, and it may lead to detrimental side effects, such as scarring and degradation of the nerve tissues at the insertion site over time.

Further, the microelectrodes can only provide electrical input and output signals to and from the native neurons. However, as mentioned hereinabove, native neurons communicate with each other not only through electrical synapses, but also through chemical-based synapses. Therefore, the electrical input and output signals provided by the microelectrodes may not be able to mimic the native nerve impulses that are transmitted through chemical-based synapses. Moreover, the summation of input potentials in the native neurons is influenced not only by the type of synapse (i.e., chemical vs. electrical), but also by the temporal pattern of action potentials arriving at the synapse, the type of pre- and/or post-synaptic neurons, the type of short-term plasticity present at the chemical synapse under stimulation, and the location of the synapse on the postsynaptic neuron. The specific neuronal targets of the microelectrodes are arbitrary, and the electrical signals provided by the microelectrodes are arbitrarily pulsed, and therefore stimulation with microelectrodes cannot match the specificity, patterns, and locations of the native synapses.

There is therefore a continuing need for improved devices and methods for accessing native neurons at target regions of the nervous system of an animal. More specifically, there is a need for devices and methods that enables neuron-specific, non-invasive access to native neurons and provides input and output signals to and from native neurons that more closely mimic the native nerve impulses.

### SUMMARY OF THE INVENTION

The present invention employs artificial neural mediators (ANMs) to provide access to native neurons at specific regions of the nervous system of an animal.

Specifically, ANMs are neural cells (either differentiated or undifferentiated) that are artificially cultivated outside of the nervous system, e.g., from neural stem cells. An individual ANM can be used to form a specific neural connection with a native neuron or collection of native neurons located at a specific region of the nervous system by guided axonal growth. The individual ANM is then connected, either directly or indirectly, to a stimulating device or a recording device. In this manner, artificial stimuli can be delivered to the native neuron or collection of native neurons by a stimulating device through the individual ANM. Alternatively, signals representing neural activities of the native neuron or collection of native neurons can be sensed by the individual ANM and sent to a recording device. Such an individual ANM therefore provides a biological or neural interface between the native neurons and the stimulating or recording device, which closely mimics natural neural signal transmission for more accurate monitoring or stimulation of the native neurons.

The ANMs of the present invention can establish a one-to-one contact with native neurons or collections of native neurons. Correspondingly, such ANMs are particularly suitable for neuron-specific monitoring and/or recording of the native neurons in the nervous system. Additionally, the ANMs of the present invention can be introduced into the nervous system in a manner that is significantly less invasive than the conventional microelectrodes, thereby reducing the risks of side effects, e.g., scarring and degradation of the nerve tissues over time. Further, the ANMs of the present invention can be readily used for applying traditional sensory stimuli/inputs to native neurons through the ANMs for deep brain stimulation. Arbitrary stimuli (such as electrical, optical, or chemical stimuli) can be converted by the ANMs into native stimuli (such as excitatory, inhibitory, and modulatory stimuli) and then delivered to the native neurons. Finally, the ANMs of the present invention provide channels for delivery of non-native chemicals or biological materials, such as biomarkers, fluorescent dyes, quantum dots, transgenic cells or tissues, etc., into the native neurons for bioengineered therapies, which cannot be achieved using conventional microelectrodes.

In one aspect, the present invention relates to a method for accessing native neurons in the nervous system of an animal, comprising:
forming one or more artificial neural mediators (ANMs) comprising neural cells;
forming a neural connection between the one or more ANMs and one or more native neurons or collections of native neurons located in the nervous system through guided axonal growth; and
accessing the one or more native neurons or collections of native neurons through said one or more ANMs.

In a specific embodiment of the present invention, the neural connection is formed by guided axonal growth of an axon from one of the ANMs into contact with one of the native neurons or collections of native neurons. For example, one of the ANMs may comprise a cell body that is located outside of the nervous system and an axon of the ANM that extends from outside of the nervous system into contact with one of the native neurons or collections of native neurons located in the nervous system. Alternatively, the axon of the ANM may extend from outside of the nervous system into contact with another ANM, which is also located in the nervous system and which comprises an axon that extends into contact with one of the native neurons or collections of native neurons.

In another embodiment of the present invention, the neural connection is formed by guided growth of an axon from one of the native neurons or collections of native neurons into contact with one of the ANMs. For example, one of the ANMs may comprise a cell body that is located outside of the nervous system and an axon that is terminated “blindly” within an external recording device, and the axon from the native neuron or collection of native neurons extends from inside the nervous system into contact with the cell body of the ANM outside of the nervous system. Alternatively, the ANM may comprise a cell body that is located inside the nervous system. The axon from the native neuron or collection of native neurons extends into contact with the cell body of such an ANM, while an axon from the ANM extends through the nervous system either into contact with an external recording device located outside of the nervous system or into contact with another ANM located outside of the nervous system.

The one or more ANMs as described hereinabove can be connected with a stimulating device so that artificial stimuli can be delivered by such a stimulating device to the one or more native neurons or collections of native neurons through the ANMs. Such ANMs can also be connected with a recording device, so that neural activities of the one or more native neurons or collections of native neurons can be recorded by the recording device through the ANMs. Further, the ANMs can be connected with a delivery device so that biological or chemical materials can be delivered to the native neurons or collections of native neurons through the ANMs.

The guided axonal growth as mentioned hereinabove can be effectuated by various means. Preferably, it is effectuated by one or more neurotrophic factors, which can be introduced by implanted medical devices, germ line modifications, and/or xenotransplants.

In a particularly preferred, but not necessary, embodiment of the present invention, an individual ANM is placed in a medical device that comprises a base and an elongated stem extending away from the base. The cell body of the individual ANM may locate in the base, and the axon of the individual ANM may locate in the elongated stem. The medical device may contain a biocompatible polymeric matrix that either is impregnated with one or more neurotrophic factors capable of effectuating cell differentiation and guided axonal growth or is selectively permeable to one or more neurotrophic factors.

In another aspect, the present invention relates to a method for treating a target region in the nerve system of an animal, comprising:
forming an ANM that comprises a neural cell;
forming a neural connection between the ANM and a native neuron or collection of native neurons located at the target region of the nervous system through guided axonal growth; and
treating the target region of the nervous system by delivering artificial stimuli or chemical or biological materials to the native neuron or collection of native neurons at the target region of the nervous system through the ANM.

The artificial stimuli that are delivered to the native neuron or collection of native neurons can be selected from the group consisting of mechanical stimuli, electrical stimuli, audio stimuli, optical stimuli, chemical stimuli, and biological stimuli. The chemical or biological materials can be selected from the group consisting of therapeutic agents, biological markers, fluorescent dyes, neurotransmitters, peptides, proteins, nucleotides, hormones, and ions.

In a further aspect, the present invention relates to a system comprising:
one or more artificial neural mediators (ANMs) each comprising a neural cell, the one or more ANMs having a neural connection with one or more native neurons or collections of native neurons located in the nervous system of an animal; and
a stimulating, recording, or delivering device connected with the one or more ANMs for recording neural activities of or for delivering stimuli or biological or chemical materials to the one or more native neurons or collections of native neurons in the nervous system through the one or more ANMs.

The system as described hereinabove may further comprise a computational device connected with the stimulating, recording, or delivering device through a communication channel for receiving signals that correlate with the neural activities of the one or more native neurons or collections of native neurons, or for controlling delivery of artificial stimuli or chemical or biological materials to the one or more native neurons or collections of native neurons in the nerve system.

More specifically, the above-described system may comprise:
one or more first ANMs having neural connection with a first native neuron or collection of native neurons located in the nervous system;
a recording device connected with the one or more first ANMs for recording neural activities of the first native neuron or collection of native neurons through the one or more first ANMs;
one or more second ANMs having neural connection with a second native neuron or collection of native neurons located in the nervous system;
a stimulating or delivery device connected with the one or more second ANMs for delivering stimuli or biological or chemical materials to the second native neuron or collection of native neurons through the one or more second ANMs; and
a computational device connected with the recording and delivery devices, wherein the computational device is constructed and arranged for receiving and processing signals from the recording device that correlate with the neural activities of the first native neuron or collection of native neurons and for controlling delivery of stimuli or biological or chemical materials to the second native neuron or collection of native neurons through the first and second ANMs.

The system may comprise multiple first ANMs arranged in a serial or parallel connection. Independently, the system may comprise multiple second ANMs arranged in either a serial or parallel connection.

In yet another aspect, the present invention relates to a medical device that comprises a base and an elongated stem extending away from the base. An ANM comprising a neural cell is located in such a medical device, while the ANM comprises a cell body located in the base of the medical device and an axon located in the elongated stem thereof.

Other aspects, features and advantages of the invention will be more fully apparent from the ensuing disclosure and appended claims.

### BRIEF DESCRIPTION OF THE DRAWINGS

FIG. 1 shows a cross-sectional view of a human brain that contains ANMs having established neural connections with native neurons in the human brain via guided axonal growth, according to one embodiment of the present invention.

FIG. 2 shows a cross-sectional view of a human brain containing ANMs that are introduced by implantable or insertable medical devices.

FIG. 3 shows a cross-sectional view of a human brain containing ANMs introduced by germ-line modifications.

FIG. 4 shows a schematic view of a system that can be used for recording neural activities of native neurons and for delivering artificial stimuli or chemical or biological materials to the native neurons in a human brain.

### DEFINITIONS

The term “native” as used here in conjunction with neurons or neural cells refers to naturally occurring neurons or neural cells that are generated by the nervous system of an animal, without any human intervention or modification.

The term “artificial neural mediator” or “ANM” as used herein refers to neurons or neural cells, either differentiated or un-differentiated, that are first cultivated outside of the nervous system of an animal and then used to form neural connections with the native neurons in the nervous system.

The term “neural connection” as used herein is similar to synapse, which refers to a junction across which nerve impulses can be passed from an axon terminal of a pre-synaptic neuron to a post-synaptic neuron.

The term “guided axonal growth” as used herein refers to a physiological response of neurons, which involves extension of the axons of such neurons as controlled by various factors, such as the existence/absence of growth/inhibition factors, nutrients, hormones, stimuli, space, optical conditions, and electric fields in the surrounding environment, as well as by the surface morphology of the substrate that supports such neurons.

The term “polymer” or “polymeric” as used herein refers to any material, composition, structure, or article that comprises one or more polymers, which can be homopolymers, copolymers, or polymer blends.

The term “biocompatible” as used herein refers to any material, composition, structure, or article that have essentially no toxic or injurious impact on the living tissues or living systems which the material, composition, structure, or article is in contact with, and produce essentially no immunological response, in such living tissues or living systems. More particularly, the material, composition, structure, or article has essentially no adverse impact on the growth and any other desired characteristics of the cells of the living tissues or living systems that are in contact with the material, composition, structure, or article. Generally, the methods for testing the biocompatibility of a material, composition, structure, or article is well known in the art.

The term “biodegradable” as used herein refers to any material, composition, structure, or article that will degrade over time by action of enzymes, by hydrolytic reaction, and/or by similar mechanisms in the body of a living organism.

The term “biostable” as used herein refers to any material, composition, structure, or article that does not degrade over time in the body of a living organism.

### DETAILED DESCRIPTION OF THE INVENTION AND PREFERRED EMBODIMENTS

The present invention will be described below in the context of illustrative examples for accessing native neurons in a human brain through artificial neural mediators (ANMs). However, it is to be understood that the teachings of the present invention are not limited to the human brain, but are also applicable to other components of the human nervous system or the nervous systems of other animals.

As mentioned hereinabove, ANMs are neural cells, either differentiated or undifferentiated, that are artificially cultivated outside of the nervous system from neural stem cells.

Neural stem cells (NSCs) are relatively primordial, undifferentiated neural cells that exist mostly in a developing (and sometimes adult) nervous system. The NSCs are responsible for subsequently forming more specialized neurons in an adult nervous system. The NSCs are operationally defined by their abilities to: (a) differentiate into cells of all neural lineages in multiple regional and developmental contexts (i.e., they are multi-potent); (b) self-renew to form new NSCs with similar multi-potency; and (c) populate developing and/or degenerating regions in the nervous system.

NSCs have been successfully isolated from the embryonic, neonatal and adult rodent nervous system, and they have also been propagated in vitro by a variety of epigenetic and genetic means, which are equally effective and safe. Epigenetic propagation of the NSCs can be achieved using various mitogens, such as epidermal growth factor (EGF), basic fibroblast growth factor (bFGF), or membrane substrates. Genetic propagation of the NSCs can be achieved using propagating genes, such as vmyc or SV40 large T-antigen. It has been shown that maintaining the NSCs in a proliferative state in culture does not subvert their ability to respond to normal developmental cues in vivo following transplantation. In other words, the NSCs can still withdraw from the cell cycle, interact with host cells, and differentiate appropriately. These extremely plastic neural cells migrate and differentiate in a temporally and regionally appropriate manner, particularly following implantation into germinal zones throughout the nervous system of the host. They participate in normal development along the neuroaxis, intermingle non-disruptively with endogenous progenitors, respond similarly to local micro-environmental cues for their phenotype determination, and appropriately differentiate into diverse neural and glial cell types. In addition, the NSCs can express foreign genes (both reporter genes and therapeutic genes) in vivo and are capable of specific neural cell replacement in the setting of absence or degeneration of neurons and/or glial cells.

The NSCs therefore can be used for forming the ANMs of the present invention. Such NSCs can be differentiated in situ, i.e., differentiation is not initiated until after introduction of the NSCs into the nervous system, which allows the NSCs to differentiate based on the chemical and/or substrate cues in the environment surrounding the target native neurons. In this manner, the NSCs form neural connections or synapses with the native neurons at the same time as they differentiate. Such in situ differentiated NSCs have a highly diverse potency, and the resulting ANMs can therefore be screened for various different therapeutic effects.

As mentioned hereinabove, guided axonal growth is used to form specific neural connections between specific ANMs and specific native neurons or collections of native neurons at a target region of the nervous system. Guided axonal growth as mentioned herein refers to a physiological response of neurons, which involves extension of the axons of such neurons as controlled by various factors, such as the existence/absence of neurotrophic factors, nutrients, hormones, stimuli, space, optical conditions, and electric fields in the surrounding environment, as well as by the surface morphology of the substrate that supports such neurons.

In a preferred, but not necessary embodiment of the present invention, neurotrophic factors (either growth enhancing or inhibitory) are used to effectuate guided axonal growth from the ANMs toward the native neurons or from the native neurons toward the ANMs. Suitable neurotrophic factors include, but are not limited to: netrins, integrins, cadherins, cytokines, insulin-like growth factor (IGF), gill-cell line-derived neurotrophic factor (GDNF), brain-derived neurotrophic factor (BDNF), platelet-derived growth factor (PDGF), vascular endothelial growth factors (VEGF), ciliary's neurotrophic factor (CNTF), epidermal growth factor (EGF), fibroblast growth factors (FGF), NT-3, and cell adhesion molecules N-CAM and L-CAM.

Physical stimulations, such as electrical or optical stimulations, can also be used to effectuate guided axonal growth from the ANMs toward the native neurons or from the native neurons toward the ANMs. Further, the guided axonal growth can be effectuated by topographically structured surfaces, such as elongated polymeric channels, microfluidic devices, or tubular guiding structures. Such topographically structured surfaces provide mechanical support for the axonal growth of the ANMs or the native neurons along specific directions that are determined by the topographic structures of such surfaces.

Preferably, but not necessarily, the ANMs comprise in situ differentiated NSCs, and the artificial axonal guidance is achieved while the NSCs are going through a specific differentiation stage, such as the neurite outgrowth stage.

Once the neural connections are established between the ANMs and the native neurons, one or more of the ANMs can then be connected, either directly or indirectly, to a stimulating or recording device. In this manner, artificial stimuli can be delivered to the native neurons or collections of native neurons by the stimulating or recording device through the ANMs, or signals representing neural activities of the native neurons or collections of native neurons can be sensed by the ANMs and sent back to the stimulating or recording device.

The stimulating or recording devices of the present invention may comprise one or more microelectrodes constructed of any suitable conductive material that is also biocompatible.

Preferably, the microelectrodes are formed of glass, carbon fibers, platinum, palladium, gold, silver, silver chloride, aluminum, chromium, tin, indium, indium tin oxide, zinc oxide, colloidal stamped carbon, electrically conductive polymers, or the like. Each microelectrode preferably has a diameter ranging from about 10 microns to about 50 microns and a length ranging from about 1 mm to about 10 mm. A voltage or current can then be applied to, or received from, one or more ANMs of the present invention through the microelectrodes for stimulating or monitoring the native neurons with which the ANMs have established neural connections.

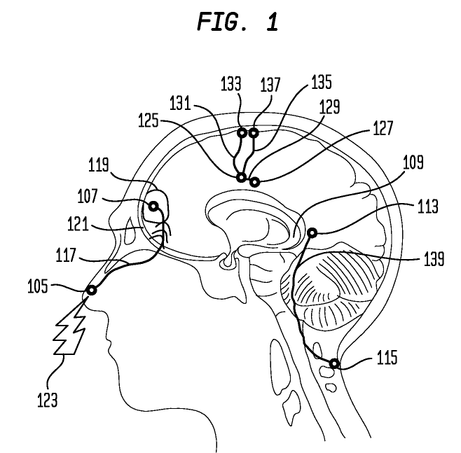

FIG. 1 shows neural connections formed between artificial neural mediators (ANMs) and native neurons in specific regions of a human brain by guided axonal growth.

A single ANM 105, which may comprises a neural stem cell or any other differentiated or undifferentiated neural cell, is located outside of a human brain, and its axon 117 is guided to grow toward and into contact with a single native neuron or collection of native neurons 107 that is located in a target brain region 119 of the human brain. For example, the target brain region 119 can be the anterior cingulate cortex, which plays an important role in a wide variety of autonomic functions, such as regulating heart rate and blood pressure.

The ANM 105 can be artificially introduced into the human brain by medical devices or by germ-line modifications, which are to be described in detail hereinafter. The guided growth of the axon 117 of the ANM 105 is effectuated by neurotrophic factor(s) 121. Upon differentiation and guided axonal growth, the ANM 105 forms a neural connection with the native neuron 107, which can be either a general or specific cell type.

After the neural connection has been established between the ANM 105 and the native neuron 107, an external stimulus 123, which can be any type of mechanical, electrical, audio, optical, chemical, or biological stimulus, can be applied by a stimulating device (not shown) to the ANM 105. The ANM 105 correspondingly applies a mediated stimulus, which is expressed in the form of a natural neuron-to-neuron synapses, to the native neuron 107.

Further, one or more chemical or biological materials, such as therapeutic agents, biological markers, fluorescent dyes, neurotransmitters, peptides, proteins, nucleotides, hormones, and ions, can be delivered by the ANM 105 to the native neuron 107. Such chemical or biological materials can be natural products or by-products of the cellular processes in the ANM 105, or they can be bio-engineered products formed by expressions of certain foreign genes in the ANM 105. Release of such chemical or biological materials by the ANM 105 may occur naturally without any human intervention, or they can be triggered by application of certain artificial stimuli to the ANM 105.

Note that in the neural connection established between the ANM 105 and the native neuron 107, neural signals can flow only in one direction, i.e., from the ANM 105 through its axon 117 to the native neuron 107, but not in the other direction, i.e., from the native neuron 107 to the ANM 105. Therefore, the neural connection between the ANM 105 and the native neuron 107 is suitable for delivering stimuli to the native neuron 107 through the ANM 105, but it cannot be used for monitoring or recording purpose, because monitoring or recording the neural activities of the native neuron 107 requires signal flow in the other direction, i.e., from the native neuron 107 to the ANM 105.

In order to enable signal flow in the other direction, i.e., from a native neuron to an ANM, a different neural connection has to be establish. Correspondingly, an ANM 125 is provided and then grafted inside a target brain region, as shown in FIG. 1.

On one hand, the ANM 125 is differentiated so that a native neuron or collection of native neurons 127 of a general or specific cell type develops a connection with the ANM 125 by guided axonal growth, i.e., an axon 129 from the native neuron 127 grows toward and into contact with the ANM 125. On the other hand, the ANM 125 has an axon 131 that is grown toward and into contact with another ANM 133 via guided axonal growth. The ANM 133 is also grafted in the human brain, but it is located at a more accessible region of the human brain, a shown in FIG. 1. Alternatively, the ANM 125 may have an axon 135 that is grown toward and into contact with another native neuron or collection of neurons 137 of a general or specific cell type, which is located at a more accessible region of the human brain, as shown in FIG. 1. Further, the ANM 133 may have an axon (not shown) that extends outside of the human brain and terminates “blindly” within an external recording device (not shown).

In this manner, signals can flow from the native neuron 127 through its axon 129 to the grafted ANM 125, and then through the axon 131 or 135 of the ANM 125 to the ANM 133 or the native neuron 137. Correspondingly, the neural activities of the native neuron 127 can be monitored by observing the neural activities of the more accessible ANM 133 or native neuron 137.

Note that ANM 125 can also be artificially “backfired”, i.e., it can be artificially stimulated through its axon at the surface to create action potentials in ANM 125 and its axon collaterals inside the brain, thus stimulating native neurons connected to the cell body, dendrites, and/or axon collaterals (not shown) of 125.

Further, a native neuron 113 located deep inside the brain may have an axon 139 that is grown toward and into contact with an easily accessible ANM 115 for germ-line modifications.

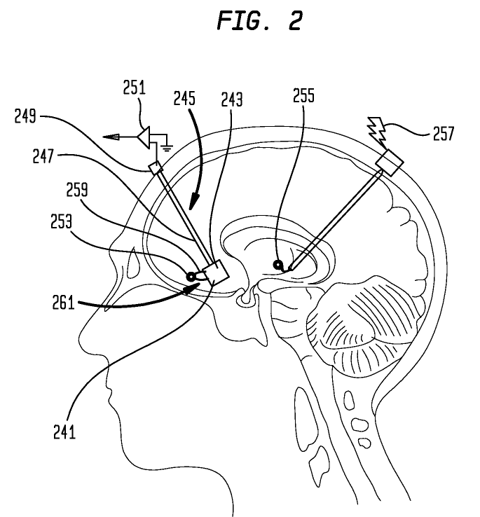

FIG. 2 shows two configurations of a medical device that can be used for introducing ANMs into a human brain. Specifically, the medical device comprises a base 241 and an elongated stem 247 that extends away from the base 241.

Such a medical device may comprise any suitable material or materials that are compatible with the attachment and growth of neural cells, such as glass, ceramic, silicon or silicon-containing compounds and mixtures, metals, and polymers. In a particularly preferred, but not necessary, embodiment of the present invention, the medical device comprises a matrix that is formed by a biocompatible polymer. More preferably, the biocompatible polymer is biostable. Biostable polymers that are suitable for use in this invention include, but are not limited to: polyurethane, silicones, polyesters, polyolefins, polyamides, poly(esteramide), polycaprolactam, polyimide, polyvinyl chloride, polyvinyl methyl ether, polyvinyl alcohol, acrylic polymers and copolymers, polyacrylonitrile; polystyrene copolymers of vinyl monomers with olefins (such as styrene acrylonitrile copolymers, ethylene methyl methacrylate copolymers, ethylene vinyl acetate), polyethers, rayons, cellulosics (such as cellulose acetate, cellulose nitrate, cellulose propionate, etc.), parylene and derivatives thereof; and mixtures and copolymers of the foregoing. Alternatively, the biocompatible polymeric material is biodegradable. Biodegradable polymers that can be used in this invention include, but are not limited to: poly(L-lactic acid), poly(DL-lactic acid), polycaprolactone, poly(hydroxy butyrate), polyglycolide, poly(diaxanone), poly(hydroxy valerate), polyorthoester; copolymers such as poly (lactide-co-glycolide), poly(hydroxy butyrate-co-valerate), poly(glycolide-co-trimethylene carbonate); polyanhydrides; polyphosphoester; poly(phosphoester-urethane); poly(amino acids); polycyanoacrylates; biomolecules such as fibrin, fibrinogen, cellulose, starch, collagen and hyaluronic acid; and mixtures and copolymers of the foregoing.

The base 241 of the medical device may have any shape or configuration, as long as it can accommodate and support the cell body of an ANM 243. Preferably, the base 241 of the medical device contains a cell medium that is suitable for culturing either differentiated or undifferentiated neural stem cells. For example, a cell medium comprising 85% high-glucose DMEM (Dulbecco Modified Eagle's Minimal Essential Medium), 10% heat-inactivated horse serum, and 5% fetal bovine serum (FBS) can be used in the base 241. Such a cell medium may further comprise a nerve growth factor (NGF). Other cell medium compositions, which may include one or more components selected from the group consisting of serum, serum substitutes, growth factors, hormones, and/or therapeutic agents, can also be used in the present invention.

The stem 247 of the medical device preferably has a tubular or cylindrical configuration with an open or close lumen therein. The axon 245 of the ANM 243 can be placed in and supported by the stem 247, and the growth direction of the axon 245 is therefore determined by the topography of the stem 247.

The ANM 243 as mentioned hereinabove is preferably grown from a neural stem cell or a collection of neural stem cells or any other progenitor cell. Differentiation and guided axonal growth of the ANM 243 can be effectuated by introducing one or more biological or chemical materials into the base 241 and/or the stem 247 of the above-described medical device. Preferably, the biocompatible polymeric matrix that forms the medical device is impregnated with one or more neurotrophic factors at the stem region 247. Such neurotrophic factors function to effectuate guided growth of the axon 245 of the ANM 243. Alternatively, the stem 247 of the medical device can comprise a porous, selectively permeable polymeric material that allows neurotrophic factors and other molecules to diffuse from the surrounding environment into the lumen of the stem 247 to effectuate guided axonal growth of the ANM 243. The term “selectively permeable” as used herein refers to materials that allow the exchange of nutrients and other metabolites therethrough. Preferably, but not necessarily, the selective permeable materials used in the present invention have an average permeability ranging from about 5,000 to about 200,000 Daltons, and more preferably from about 50,000 to about 150,000 Daltons.

In a specific embodiment of the present invention, the base 241 of the medical device is implanted into the brain region of interest for recording of the neural activities therein, as shown at the left side of FIG. 2. Upon differentiation, a native neuron 253 of general or specific type grows an axon 261 toward and into contact with the ANM 243 that is located in the base 241 of the implanted medical device. The ANM 243 can also grow an axon or axon collateral 259 toward and into contact with the native neuron 253. The axon 245 of the ANM 243 extends through the stem 247 of the medical device to outside of the human brain and terminates within a recording chamber 249, which is connected to a recording device 251 (e.g., an operational amplifier). In this manner, neural activities of the native neuron 253 can be recorded by the recording device 251 through the ANM 243 that is located in the implanted medical device.

Further, the above-described medical device can be used to support ANMs through which artificial stimuli can be delivered to native neurons in the human brain. Specifically, the base of such a medical device is located outside of the human brain, while the stem of the medical device is inserted into a deep brain region (e.g., the thalamus) in the human brain, as shown at the right side of FIG. 2. An ANM can be first grown in the base of the medical device and then induced to undergo guided axonal growth to grow an axon through the stem of the medical device into the deep brain region. The axon of such an ANM forms a neural connection with a native neuron 255 located at the deep brain region, as shown in FIG. 2. An artificial stimulus 257 (e.g., an electrical stimulus) can be applied to the ANM which, in turn, causes neurotransmission from the ANM to the native neuron 255 in the deep brain region, thereby stimulating the native neuron 255.

The ANMs of the present invention can be introduced into the human brain by a medical device as described hereinabove, and they can also be introduced into the human brain by germ line modifications (such as transgenic animals, artificial chromosomes, and/or genetic engineering) or tissue graft.

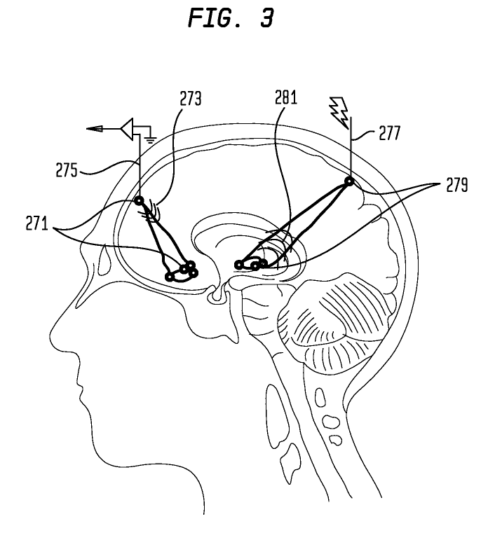

FIG. 3 shows two configurations of ANMs 271 and 279, which have been introduced into the human brain by means of germ line modifications or tissue graft. On one hand, electrode(s) 275 or other recording device can be used to record from the ANMs 271. On the other hand, electrode(s) 277 or other stimulation device can be used to stimulate the ANMs 279. Axons from the ANMs 271 and 279 are guided toward other ANMs and/or native neurons in other brain regions by neurotrophic factors 273 and 281, which are generated by other ANMs and/or native neurons. Because the ANMs 271 and 279 are connected with specific ANMs and/or native neurons in other brain regions, the ANMs 271 and 279 enables mediated neuron-specific recordation from, or stimulation of, such other brain regions.

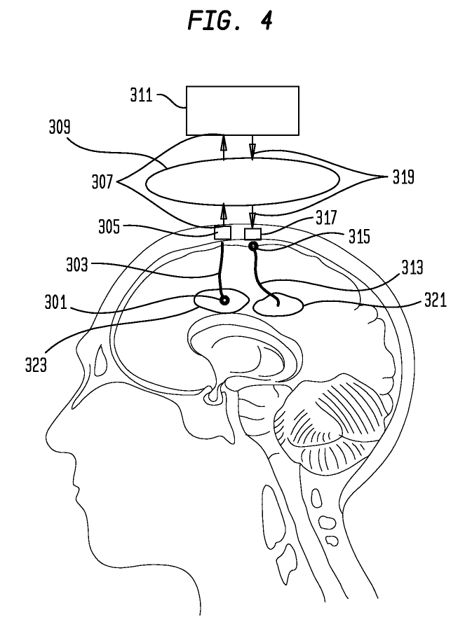

FIG. 4 shows a system for simulating native neurons and recording from native neurons located in the human brain through ANMs.

Specifically, one or more transducers 305 sense neural activity in the axon 303 of an ANM 301, which is located in a specific brain region 323 and has a neural connection with a native neuron (not shown) in the brain region 323. The transducers 305 correspondingly transform the sensed neural activity into a signal 307 of an appropriate nature and format. The signal 307 can be an electrical, optical, chemical, or biological signal, and it may have either analog or digital format. Such a signal 307 is then transmitted through a communication channel 309 of an appropriate nature (e.g., wire, wireless, optical, chemical, etc.) to a computational device 311. The computational device 311 may comprise a computer, central processor unit (CPU), microprocessor, or integrated circuitry, which is constructed and arranged to process and analyze signals received from the communication channel 309.

Similarly, the computational device 311 can generate and send a signal 319 through the communication channel 309 to one or more actuators 317 which, in turn, stimulates an ANM 315. The stimulation applied by the actuators 317 is delivered to a specific brain region 321 through an axon of the ANM 321, which is located in the specific brain region 321 and has formed a neural connection with one or more native neurons located in the brain region 321.

Although FIG. 4 illustratively shows a single ANM 301 for native neural activity sensing and a single ANM 315 for stimulation delivery, it is understood that the system of the present invention can comprise any number of ANMs for native neural activity sensing and any number of ANMs for stimulation delivery. For example, multiple ANMs arranged either in a serial or parallel connection can be used for native neural activity sensing or for stimulation delivery.

The present invention can be used for treating any diseases associated with the nervous system of an animal. For example, the present invention can be used for treatment of neurodegenerative diseases, neurological and psychiatric disorders, or brain injuries. The present invention can also be used for delivery of therapeutic agents, biological markers, or chemical compounds (either natively generated or artificially introduced) through the ANMs of the present invention to native neurons that are connected with the ANMs of the present invention. The present invention can further be used to achieve brain modification through artificial brain stimulation. The brain modification may involve increase of attention, induction of insomnia, sensory replacement, or increase of sensory capacity in humans and/or animals. The brain modification may also involve enhanced training, communication, or control in animals. The term “treat” or “treating” as used herein broadly covers the performance of therapeutic treatment or other types of modification to the nervous system.

The ANMs of the present invention can be introduced either in a permanent manner or in a temporary manner, depending on the specific treatment requirements of the disease. For example, chronic brain stimulation or long-term delivery of therapeutic agents is necessary for treating certain chronic neurodegenerative diseases, such as Parkinson's disease, Alzheimer's disease, Huntington's disease, and amyotrophic lateral sclerosis (ALS). Accordingly, permanent introduction of the ANMs is provided. However, temporary brain stimulation is effective in achieving regional neuron cell regeneration for treating acute brain or spinal cord injuries. In such events, the ANMs can be introduced only for a limited time to achieve the desired therapeutic effect, and subsequently, the ANMs can either be allowed to degrade naturally or be artificially terminated/removed.

Still further, the present invention can be used to monitor brain activity for the purpose of medical diagnostics (e.g., diagnosis of epilepsy) or for providing communication between the brain and an external system (e.g., transmitting brain commands to medical prosthetics).

While the present invention has been particularly shown and described with respect to preferred embodiments thereof, it will be understood by those skilled in the art that the foregoing and other changes in forms and details may be made without departing from the spirit and scope of the present invention. It is therefore intended that the present invention not be limited to the exact forms and details described and illustrated, but fall within the scope of the appended claims.

What is claimed is:

1. A method for accessing native neurons in the nervous system of an animal, comprising:
forming one or more artificial neural mediators (ANMs) comprising neural cells;
forming a neural connection between said one or more ANMs and one or more native neurons or collections of native neurons located in the nervous system through guided axonal growth; and
accessing the one or more native neurons or collections of native neurons through said one or more ANMs.

2. The method of claim 1, wherein the neural connection is formed by guided growth of an axon from one of the ANMs into contact with one of the native neurons or collections of native neurons.

3. The method of claim 2, wherein said one of the ANMs comprises a cell body that is located outside of the nervous system, and wherein the axon of said one of the ANMs extends from outside of the nervous system into contact with said one of the native neurons or collections of native neurons or another ANM located in the nervous system.

4. The method of claim 3, wherein the axon of said one of the ANMs extends from outside of the nervous system into contact with another ANM located in the nervous system, and wherein said another ANM comprises an axon that extends into contact with said one of the native neurons or collections of native neurons.

5. The method of claim 1, wherein the neural connection is formed by guided growth of an axon from one of the native neurons or collections of native neurons into contact with one of the ANMs.

6. The method of claim 5, wherein said one of the ANMs comprises a cell body that is located outside of the nervous system and an axon that is terminated within an external recording device, and wherein the axon from said one of the native neurons or collections of native neurons extends from inside the nervous system into contact with the cell body of said one of the ANMs located outside of the nervous system.

7. The method of claim 5, wherein said one of the ANMs comprises a cell body that is located in the nervous system and an axon that extends into contact with another ANM located outside of the nervous system, and wherein the axon from said one of the native neurons or collections of native neurons extends into contact with the cell body of said one of the ANMs.

8. The method of claim 1, wherein the one or more ANMs are connected with a stimulating device, so that artificial stimuli can be delivered by the stimulating device to said one or more native neurons or collections of native neurons through said one or more ANMs.

9. The method of claim 1, wherein the one or more ANMs are connected with a recording device, so that neural activities of said one or more native neurons or collections of native neurons can be recorded by the recording device through said one or more ANMs.

10. The method of claim 1, wherein the one or more ANMs are connected with a delivery device, so that biological or chemical materials can be delivered to said one or more native neurons or collections of native neurons through said one or more ANMs.

11. The method of claim 1, wherein the guided axonal growth is effectuated by one or more neurotrophic factors that are introduced by implanted medical devices.

12. The method of claim 1, wherein the guided axonal growth that is effectuated by one or more neurotrophic factors that are introduced by germ line modifications.

13. The method of claim 1, wherein one of the ANMs is located in a medical device that comprises a base and an elongated stem extending away from the base, wherein said one of the ANMs has a cell body located in the base of the medical device and an axon located in the elongated stem thereof, and wherein the medical device comprises a biocompatible polymeric matrix that is either impregnated with one or more neurotrophic factors capable of effectuating cell differentiation and guided axonal growth or is selectively permeable to said one or more neurotrophic factors.

14. A method for treating a target region in the nervous system of an animal, comprising:
forming an ANM that comprises a neural cell;
forming a neural connection between the ANM and a native neuron or collection of native neurons located at the target region of the nervous system through guided axonal growth; and
treating the target region of the nervous system by delivering artificial stimuli or chemical or biological materials to the native neuron or collection of native neurons at the target region of the nervous system through the ANM.

15. The method of claim 14, wherein the artificial stimuli are selected from the group consisting of mechanical stimuli, electrical stimuli, audio stimuli, optical stimuli, chemical stimuli, and biological stimuli, and wherein the chemical or biological materials are selected from the group consisting of therapeutic agents, biological marks, fluorescent dyes, neurotransmitters, peptides, proteins, nucleotides, hormones, and ions.

16. A system comprising:
one or more artificial neural mediators (ANMs) each comprising a differentiated or undifferentiated neural cell, said one or more ANMs have a neural connection with one or more native neurons or collections of native neurons located in the nervous system of an animal; and
a stimulating, recording, or delivery device connected with said one or more ANMs for recording neural activities of, or for delivering artificial stimuli or chemical or biological materials to, said one or more native neurons or collections of native neurons in the nervous system through the one or more ANMs.

17. The system of claim 16, further comprising a computational device connected with the stimulating, recording, or delivery device through a communication channel for receiving signals that correlate with the neural activities of said one or more native neurons or collections of native neurons or for controlling delivery of stimuli or biological or chemical materials to said one or more native neurons or collections of native neurons in the nervous system.

18. The system of claim 16, comprising:
one or more first ANMs having neural connection with a first native neuron or collection of native neurons located in the nervous system;
a recording device connected with said one or more first ANMs for recording neural activities of the first native neuron or collection of native neurons through the one or more first ANMs;
one or more second ANMs having neural connection with a second native neuron or collection of native neurons located in the nervous system;
a stimulating or delivery device connected with said one or more second ANMs for delivering artificial stimuli or chemical or biological materials to the second native neuron or collection of native neurons through the one or more second ANMs; and
a computational device connected with the recording and delivery devices, wherein said computational device is constructed and arranged for receiving and processing signals from the recording device that correlate with the neural activities of the first native neuron or collection of native neurons and for controlling delivery of artificial stimuli or chemical or biological materials to the second native neuron or collection of native neurons through the first and second ANMs.

19. A medical device comprising a base and an elongated stem that extends away from the base, wherein an artificial neural mediator (ANM) comprising a neural cell is located in said medical device, wherein said ANM comprises a cell body located in the base of the medical device and an axon located in the elongated stem thereof.

20. The medical device of claim 19, wherein the base and the elongated stem of said medical device comprise a biocompatible polymeric matrix that is either impregnated with one or more neurotrophic factors capable of effectuating cell differentiation and guided axonal growth or is selectively permeable to said one or more neurotrophic factors.
